(19) United States
(12) Patent Application Publication
(10) Pub. No.: US 2026/0104388 A1
(43) Pub. Date: Apr. 16, 2026

(54) BIO SENSOR HAVING THERMAL DISSIPATION VIA COIL LAYOUT

(71) Applicant: Allegro MicroSystems, LLC, Manchester, NH (US)

(72) Inventors: Guillermo Airaldi, Salinas Canelones (UY); Rémy Lassalle-Balier, Bures sur Yvette (FR); Jorge Gabriel Varela Kuolas, Montevideo (UY)

(73) Assignee: Allegro MicroSystems, LLC, Manchester, NH (US)

(21) Appl. No.: 18/915,603

(22) Filed: Oct. 15, 2024

(51) Int. Cl. G01N 27/74; G01N 33/487; G01R 33/00; G01R 33/09

(52) U.S. Cl. CPC G01N 27/74; G01N 33/487; G01R 33/0052; G01R 33/093

(57) ABSTRACT

Example embodiments provide for a magnetic field bio sensor having a first coil with first and second portions on different metal layers connected by first vias and a second coil having first and second portions on different ones of the metal layers connected by second vias. The second portion of the first coil overlaps with the first portion of the second coil to promote heat dissipation via an inactive one of the first and second coils and reduce die area.

### BACKGROUND

Magnetic field sensors utilize magnetic field sensing elements to detect one or more magnetic fields for various purposes. For example, magnetic field sensors are often used to detect a current flowing in a conductor. Magnetic field sensors may also be used to detect a ferromagnetic or conductive target and may generally act to detect motion or position of the target. Such sensors are found in many technology areas including robotics, automotive, manufacturing, biotechnology, and so forth.

Magnetoresistance (MR) elements are a class of magnetic sensing elements having a variable resistance that changes in response to changes in an applied or sensed magnetic field. There are different types of magnetoresistance elements, for example, semiconductor magnetoresistance elements such as ones including Indium Antimonide (InSb), anisotropic magnetoresistance (AMR) elements, giant magnetoresistance (GMR) elements, and tunneling magnetoresistance (TMR) elements, which are also referred to as magnetic tunnel junction (MTJ) elements.

### SUMMARY

Example embodiments of the disclosure provide methods and apparatus for an MR bio sensor having a coil configuration for thermal management and sensor layout. In embodiments, thermal management includes a coil layout configured for bio sensing and partial coil overlap to enhance heat diffusion across the full die. Coil routing can include wires behaving as heat pipes between adjacent columns for enhancing heat diffusion and dissipation. A light-blocking top level metal shield can provide further heat diffusion and dissipation.

In one aspect, a magnetic field bio sensor comprises: a first coil having first and second portions on different metal layers, wherein the first and second portions of the first coil are connected by first vias; and a second coil having first and second portions on different ones of the metal layers, wherein the first and second portions of the second coil are connected by second vias, wherein the second portion of the first coil overlaps with the first portion of the second coil to promote heat dissipation via an inactive one of the first and second coils.

A sensor can further include one or more of the following features: the first portions of the first and second coils are formed on a first one of the metal layers, the first coil is on multiples ones of the metal layers, the second portions of the first and second coils are formed on a second one of the metal layers, the second coil is on multiples ones of the metal layers, the first coil includes an active area in which a first portion of the first coil splits into N segments, the N segments are parallel to each other, the N segments recombine, N is between 2 and 100 inclusive, the active area is configured to sense return from at least one MR element proximate a bio sample, the overlap of the second portion of the first coil overlaps and the first portion of the second coil is configured to dissipate heat generated by the first coil via heat transfer in the second coil, the sensor comprises an IC package having bio pixels for respective samples of the bio material, and/or the first coil provides first and second pixels.

In another aspect, a method comprises: forming a first coil having first and second portions on different metal layers, wherein the first and second portions of the first coil are connected by first vias; and forming a second coil having first and second portions on different ones of the metal layers, wherein the first and second portions of the second coil are connected by second vias, wherein the first and second coils form part of a magnetic field bio sensor, wherein the second portion of the first coil overlaps with the first portion of the second coil to promote heat dissipation via an inactive one of the first and second coils.

A method can further include one or more of the following features: the first portions of the first and second coils are formed on a first one of the metal layers, the first coil is on multiples ones of the metal layers, the second portions of the first and second coils are formed on a second one of the metal layers, the second coil is on multiples ones of the metal layers, the first coil includes an active area in which a first portion of the first coil splits into N segments, the N segments are parallel to each other, the N segments recombine, N is between 2 and 100 inclusive, the active area is configured to sense return from at least one MR element proximate a bio sample, the overlap of the second portion of the first coil overlaps and the first portion of the second coil is configured to dissipate heat generated by the first coil via heat transfer in the second coil, the sensor comprises an IC package having bio pixels for respective samples of the bio material, and/or the first coil provides first and second pixels.

### BRIEF DESCRIPTION OF THE DRAWINGS

The foregoing features of the disclosure, as well as the disclosure itself may be more fully understood from the following detailed description of the drawings, in which:

FIG. 1 is a schematic representation of an example MR bio sensor having coil layout thermal dissipation in accordance with example embodiments of the disclosure;

FIG. 2 shows an example bridge configuration for MR elements in the sensor of FIG. 1;

FIG. 3 is a high level block diagram showing a top area for first return paths and a bottom area for second return paths, and a middle area for active paths;

FIG. 4A is a top view and FIG. 4B is a cross-sectional view along line AB in FIG. 4A of an example MR bio sensor having complementary metal levels in top and bottom layers;

FIG. 5 is a schematic representation of an example MR bio sensor having sample regions surrounded by separate coils with coil overlap; and

FIG. 6 is a partially transparent isometric view of first and second coils having some overlap;

FIG. 6A is a partially transparent isometric view of another configuration of first and second coils having some overlap;

FIG. 7A is a top view and FIG. 7B is a cross-sectional view along line A-A of a three-coil configuration 700 having coil overlap to promote heat dissipation;

FIG. 8 is a schematic representation of a coil portion separating into coil segments in an active area of the coil;

FIG. 8A is a schematic representation of a GMR separating into serpentine coil segments; and

FIG. 9 is a representation of an example MR bio sensor IC package having bio pixels and coil layout heat dissipation.

### DETAILED DESCRIPTION

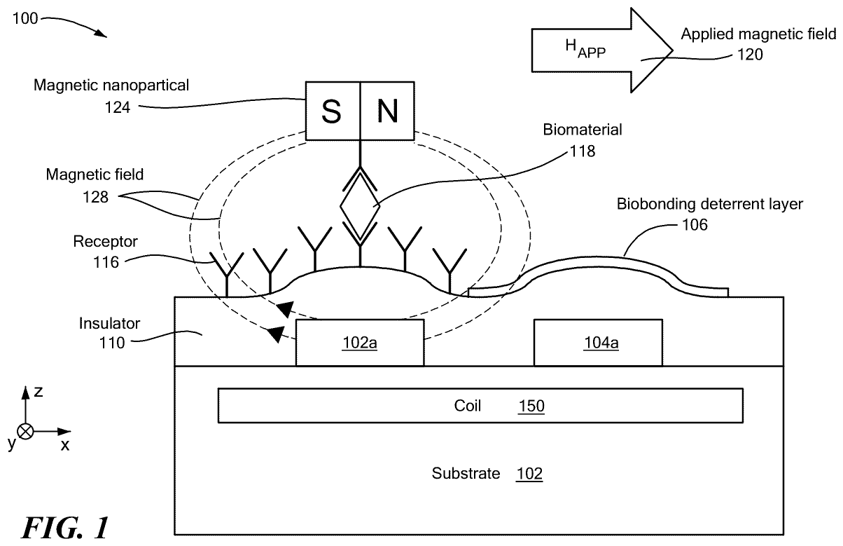

FIG. 1 is a schematic representation of example magnetic field bio sensor 100 having coil layout heat dissipation in accordance with example embodiments of the disclosure. The sensor 100 can include magnetic field sensing elements that form a detector operable to detect ferromagnetic material and thereby evaluate samples of biologic material, as described more fully below. The ferromagnetic material can be disposed over the magnetic field sensor 100, i.e., displaced in a direction parallel to a z-axis.

While example sensor embodiments may be described in conjunction with eight (8) MR elements, and more particularly with GMR elements, the general concepts and structures sought to be protected herein can be applied to sensors having other numbers of MR elements, such as one (1), two (2), three (3), or four (4), elements per pixel.

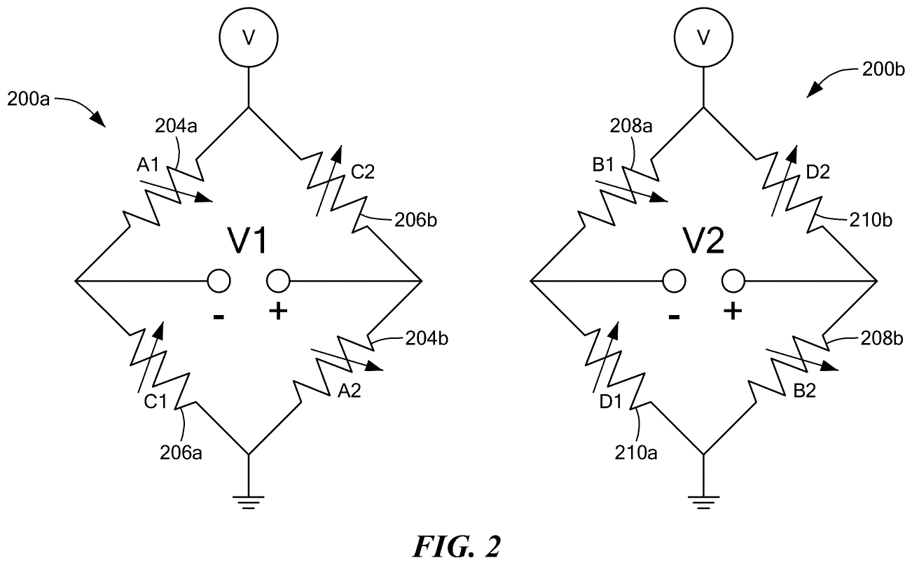

FIG. 2 shows an example configuration of eight MR elements A1, A2, B1, B2, C1, C2, D1, and D2 coupled in two bridge circuits, a first bridge circuit 200a is comprised of MR elements A1, A2, C1, and C2 represented as variable resistors 204a, 204b, 206a, and 206b, respectively. A second bridge circuit 200b is comprised of MR elements B1, B2, D1, and D2 represented as variable resistors 208a, 208b, 210a, and 210b, respectively. In example embodiments, there is one bridge per pixel.

Referring again to FIG. 1, an example magnetic-field biosensor 100 includes a substrate 102 with two (2) MR elements 102a, 104a provided on a top surface of the substrate 102. The MR elements 102a, 104a can be encapsulated in an insulator 110 that prevents oxidation of the MR elements. One or more receptors 116 are attached to the top surface of the insulator 110 above the MR element 102a. The receptors 116 can capture specific biological material, such as biomaterial 118. A biobonding deterrent layer 106 is disposed on the top surface of the insulator 110 above MR element 104a. Biobonding deterrent layer 106 can prevent any receptors from attaching thereto.

A fluid can be poured on the surface of the insulator 110. Specific biomaterial present in the fluid can be captured by the receptors 116. The sensor 100 can be later washed and a solution with one or more magnetic nanoparticles 124 (that are configured to attach to the biomaterial 118) can be poured on the sensor 100. If the biomaterial 118 is attached to one or more of the receptors 116, then magnetic nanoparticles 124 are attached to each of the biomaterial

The MNPs (Magnetic NanoParticles) attach to a bio marker. The sensor attempts to sense if the bio/marker is present. If the bio marker is absent, the MNPs remain in a colloidal stable state and do not affect the magnetic field emitted by the coil, so the bridge output is zero. If the bio marker is present, then the MNPs are concentrated at the surface of the sensor which tends to reduce the demagnetizing field inside the GMR. This increases the sensitivity of the GMR with the thin insulator (or without the deterrent layer). This makes the bridge output non-zero.

In the illustrative embodiment, the MR element 102a detects more of the magnetic field 128 from the magnetic nanoparticles 124 than does the MR element 104a. In one example, a detection of magnetic field 128 of magnetic nanoparticles 124 (and hence, the detection of the biomaterial 118) is performed by taking a difference of electrical changes of the MR element 102a and electrical changes of MR element 104a by placing MR elements 102a, 104a in a half bridge or full bridge.

The magnetic nanoparticles 124 can generate a magnetic field 128. The magnetic nanoparticles 124 behave like a super paramagnet and can be collectively configured to align with an applied magnetic field 120. Otherwise, the magnetization directions of the magnetic nanoparticles are randomly distributed. MR elements 102a, 104a may be connected in series or in parallel to form a single device used to detect magnetic field 128 from magnetic nanoparticles 124 and thereby detect biomaterial 118. In this configuration, a magnetic field measured at the MR elements 102a, 104a may be opposite to applied magnetic field 120.

In some embodiments, magnetic field 120 can be generated in the x-z plane, with the field generated near the center of coil being primarily in the direction of the z axis. Thus, the field applied to MR elements 102a, 104a may be primarily in the x-axis direction. MNPs near GMR also get field in the X direction due to the coil.

In other examples, magnetic-field biosensor 100 in FIG. 1 may be expanded. In one example, the magnetic-field biosensor may further include two more MR elements, one located under another biobonding deterrent layer and the other located under one or more additional receptors. The additional components may extend into the page of FIG. 1 or be side-by-side with the components in FIG. 1. Four (4) MR elements may be disposed in a full bridge. A differential output of the full bridge may be used to determine if magnetic nanoparticles exist.

In other examples, additional pairs of MR elements may be further expanded into the page of FIG. 1 and/or side-by-side with the components in FIG. 1 with one additional MR element in a pair located under a biobonding deterrent layer and the other located under one or more receptors. The MR elements may be disposed in a full bridge, as shown in FIG. 2. A differential output of the full bridge may be used to detect if magnetic nanoparticles exist.

As shown in the example of FIG. 1, a magnetic biosensor can include one or more excitation coils 150 configured to generate applied magnetic field 120 when excited with an electric current. In some cases, excitation coils 150 may include two coils positioned under respective ones of the two MR elements 102a, 104a. In some cases, the two coils may have parallel lines that are aligned with parallel lines of the respective MR elements 102a, 104a. Examples of coils structures and layouts that may be used within a magnetic biosensor are shown in subsequent figures.

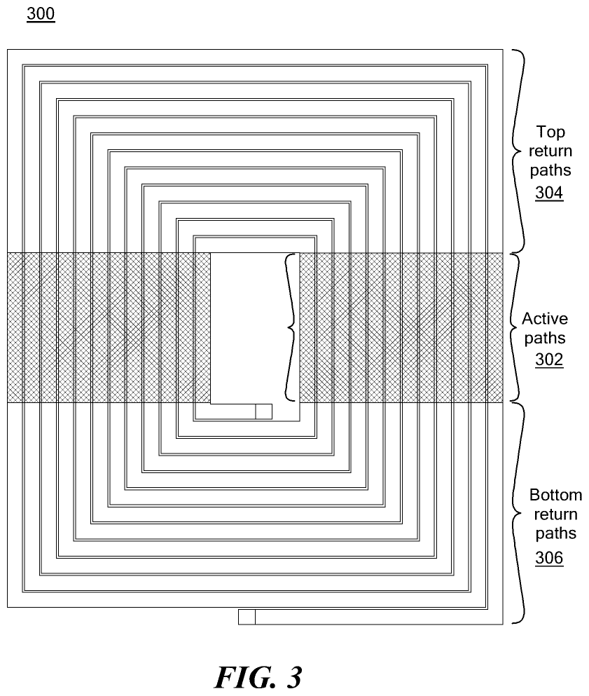

FIG. 3 shows a high level diagram of an example MR bio sensor 300 having an area 302 of active coil paths, a top area of return coil paths 304 and a bottom area of return coil paths 306. As shown and described more fully below, the physical layout of the coils in the return areas 304, 306 for bio sensing applications includes partial overlaps of coils for enhancing heat diffusion across the full die, as described more fully below. The coils operate as heat pipes between adjacent columns of coils to promote heat diffusion and dissipation. By overlapping the coils, the pitch between coil centers may be reduced to save in total die area.

It is understood that the different areas, e.g., active, top and bottom 302, 304, 306 may be separated by some small distance, however, these layers combine to form a substantially planar sensing layer configured to sense MNPs above the sensing layer. As used herein, “substantially planar” for the sensing layer refers to a distance extending from a top-most surface of any sensing element to a bottom-most sensing element. The different areas may be in the order of hundreds of microns wide/long and in the micron range for thickness (out of plane direction). It is understood that the sensing layer is sensitive to in plane field.

As will be appreciated, thermal management in bio sensors is desirable to prevent sample-destroying temperatures. For example, for some samples in biological sensors, the temperature of the biological functions on top of the heat sources in the die cannot rise above room temperature more than +5° C. Thermal management should lower average temperature value and improve thermal homogeneity across the die. Embodiments of the disclosure provide MR bio sensors having thermal management provided by coil configurations, high current drive coil paths, and/or top-level metal shield(s).

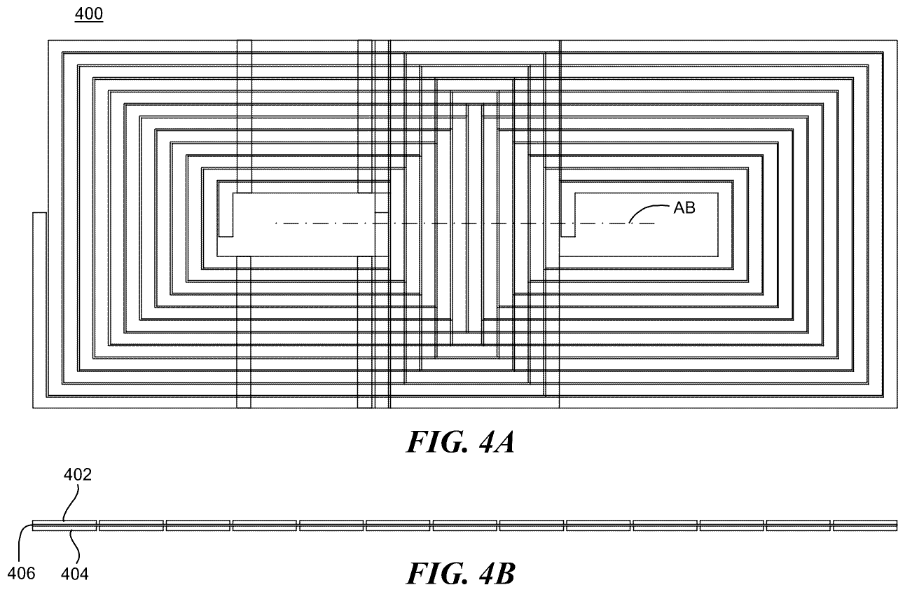

FIG. 4A is a top view and FIG. 4B is a cross-sectional view along line AB in FIG. 4A of an example MR bio sensor 400 having complementary metal levels in top and bottom layers. It is understood that the length of the line AB corresponds to what is shown in the cross-sectional view of FIG. 4B. In the illustrated embodiment, top return path coils are provided in a first metal layer 402, bottom return path coils are provided in a second metal layer 404. The first and second metal layers 402, 404 are separated by an inter metal dielectric (IMD) layer 406 that can provide via connections between the first and second metal layers 402, 404.

The illustrated return coil path configuration of FIGS. 4A and 4B enables stacking identical coils overlapping the bottom return paths of a coil with the top return paths of another coil.

In example embodiments, a lower metal level is used for the top return paths and the highest metal level is used for the bottom return paths. In some embodiments, even metal levels are used for one of the return paths groups (top or bottom) and the odd metal levels are used for the other group of return paths. In another embodiment alternating contiguous metal levels between both groups of return paths of the coil. Making a coil stackable with itself allows to have a unique coil design which helps to match each excitation results. This way, when a coil is activated, the other ones (the non-activated ones) favor the heat dissipation and reduce the settling time of itself (of the coil that will be activated in a next step). It is worth mentioning that the overlap between coils remains small enough that the coupling factor between the coils remains small. Even the non-active coils could be loaded to have null impact over the activated coil.

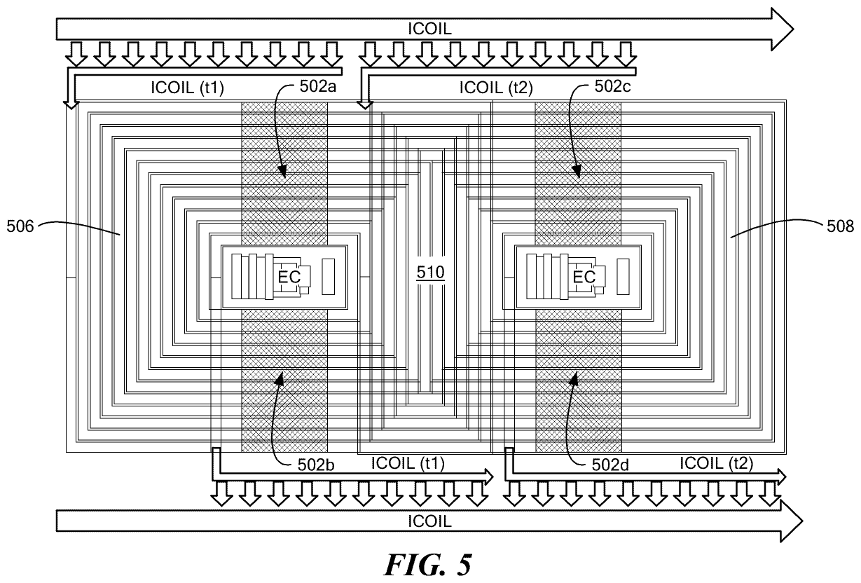

FIG. 5 shows an example bio sensor 500 having first, second, third, and fourth bio sample regions 502a-d. A first coil 506 is coupled to the first and second sample regions 502a,b and configured in rectangles of differing sizes each spaced from an adjacent one. A second coil 508 is similarly configured and connected to the third and fourth sample regions 502c,d. As can be seen, there is an overlap region 510 where the first and second coils 506, 508 overlap.

In embodiments, shields, which may comprise metal, can be placed over active electronic circuitry when highest level metal is not used, e.g., to avoid a short circuit between active top level metal paths vs top level metal shield. For example, shields can be placed over the electrical circuitry EC in the centers of the coils and in some parts of the zones identified as ICOIL, ICOIL(t1) and ICOIL(t2).

In operation, only one of the first and second coils 506,508 has current flow at a given time. In the illustrated embodiment, a first current i1 flows through the first coil 506 during a first time period t1 and a second current i2 flows through the second coil 508 during a second time period t2.

The routing of the first and second coils 506, 508 is configured to remove/reduce the presence of hot spots by minimizing the crosses of high current paths and having the active current paths close to the non-activated coils. The coil 506, 508 paths are close enough to provide heat conduction when considering that the current is stopped before the active areas so that it does not create additional field on the activated coil. The inactive parts of the coil paths remain without current to promote heat conduction.

The metal shields may be used to avoid light incidence to prevent degrading/affecting electronic circuit performance. The shields also work as a heat sink for the die for improving heat dissipation which results in a lower average temperature value and improves the thermal homogeneity across the die.

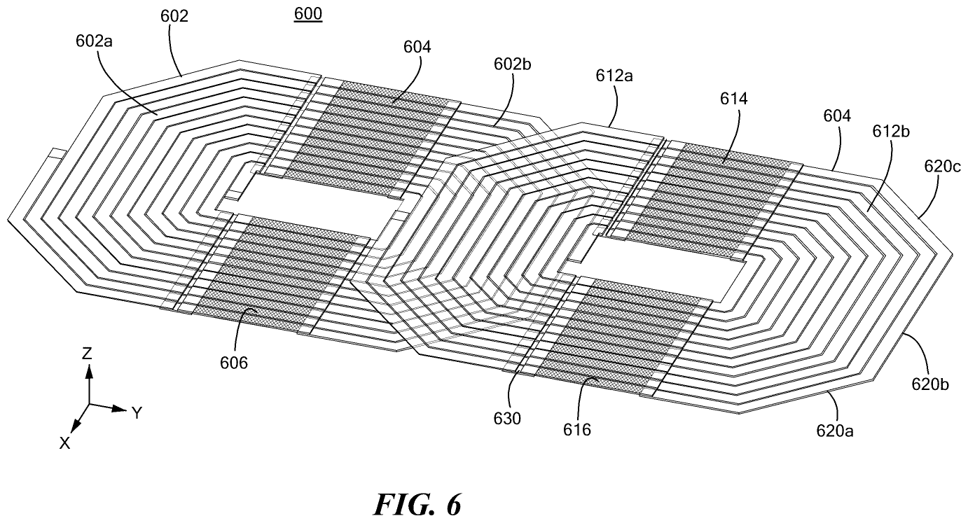

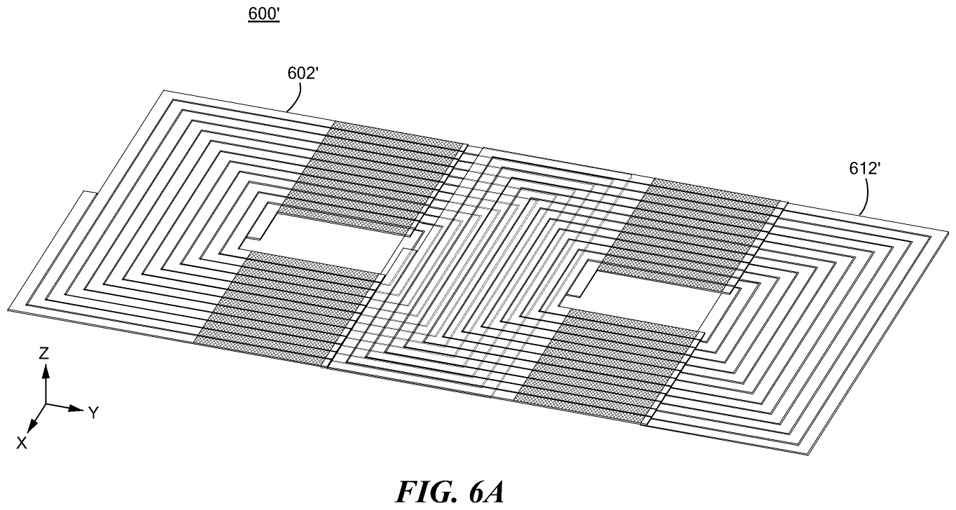

FIG. 6 shows an example coil configuration 600 providing coil overlap to promote heat dissipation via the inactive coil. A first coil 602 includes a first portion 602a on a first metal layer and a second portion 602b on a different metal layer. The first coil 602 includes first and second active areas 604, 606 configured for GMR sensing, as described above. A second coil 612 includes a first portion 612a on first metal, which may be the same metal layer as the first portion 602a of the first coil 602, and a second portion 612b on a different metal layer. The second coil 612 includes first and second active areas 614, 616.

As can be seen, the first portion 612a of the second coil and the second portion 602b of the first coil overlap. In embodiments, only one of the first and second coils 602, 612 are active at any given time. If say, the first coil 602 is active, i.e., current flows, then the second coil 612 is not active so the second coil can transfer heat generated by the current flow in the first coil. As described above, bio samples can be damaged by heat above certain temperatures so heat dissipation via inactive coils can protect the bio samples.

In the illustrated embodiment, a coil, such as coil 612 includes three segments 620a,b,c to effect a 180 degree change in coil/current direction. It is understood that any practical number of segments having any suitable angle can be used to meet the needs of a particular application.

As described more fully below, a transition area 630 may be provided to enable cable and other electrical connections to meet the needs of a particular application.

In example embodiments, each active area provides one pixel and each coil provides two pixels.

FIG. 6A shows another example coil configuration 600′ providing coil overlap to promote heat dissipation via the inactive coil similar to that shown in FIG. 6 but with first and second coils 602′, 612′ that are square.

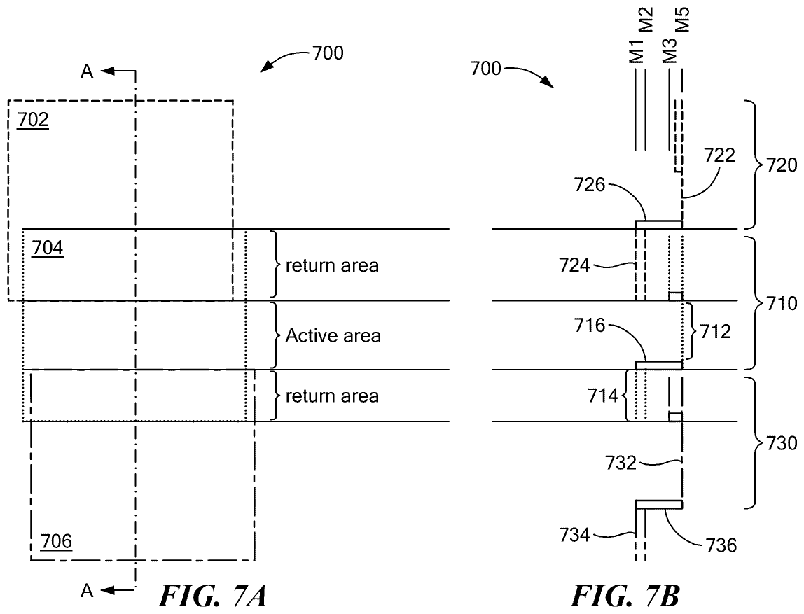

FIG. 7A is a top view and FIG. 7B is a cross-sectional view along line A-A of a three-coil configuration 700 having coil overlap to promote heat dissipation. First, second, and third coils 702, 704, 706 are arranged to partially overlap in a manner similar to that shown in FIG. 6A.

The second coil 704 includes a first portion 710 of which an active area 712 forms a part, and a second portion 714. The active area 712 is closest to the bio sample (not shown) At least one via 716 electrically connects the first and second portions 710, 714 of the coil which are located on different metal layers.

Similarly, the first coil 702 includes first and second portions 720, 724 on different metal layers and an active area 722, and the third coil 706 includes first and second portions 730, 734 on different metal layers and an active area 732.

In an example, embodiment metal layers M1, M2, M3, MS (FIG. 7B) can be used to form the coils. It is understood that any suitable stackup scheme can be used to meet the needs of a particular application in which overlapping coils are desirable.

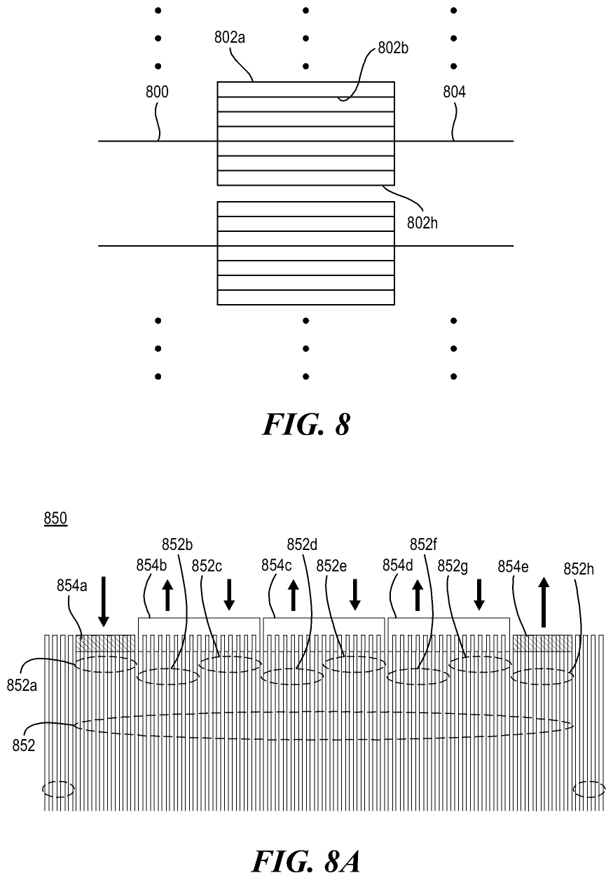

FIG. 8 shows an example coil configuration for an active area, such as the first active area 604 of FIG. 6. In the illustrated embodiment, a first coil portion 800 splits into a number of segments 802a-h having a smaller cross-sectional area than the source coil portion 800. The segments 802a-h can connect to a second coil portion 804 that may be similar to the first coil portion 800. Similarly, other coil portions can split into segments in the active area and then recombine.

The coil segments 802 are configured to minimize the distance from the segments to the MR sensing elements and bio sample to maximize SNR and otherwise enhance sensing performance.

It is understood that any practical number of coil segments can be used to meet the needs of a particular application. It is further understood that any suitable geometry for the coils and the coil segments can be used to enhance sensing performance.

FIG. 8A shows an example of an active area configuration in which GMR segments are serpentine in arrangement. To facilitate an understanding of an example embodiment, FIG. 8A shows a single-element GMR structure 850 having a serpentine layout of narrow, parallel lines. The structure 850 can have an overall rectangular shape, but with protruding shorts for magnetic noise rejection. The structure 850 includes a plurality of parallel lines 852, a first plurality of metal pads 854a-e (854 generally) arranged about a first end of the structure, and a second plurality of metal pads 856a-d (856 generally) arranged along an opposite end. The parallel lines 852 may be formed using an etching process, or ion milling for example. The metal pads 854, 856 may be comb-shaped and deposited or otherwise formed onto the ends of parallel lines 852, as discussed further below.

Two of the metal pads may correspond to terminals of the element, whereas the other metal pads are provided to interconnect the parallel lines in a serpentine layout. In the example of FIG. 8A, metal pads 854a and 854e correspond to the terminals, whereas metal pads 854b-d, 856a-d are provided to achieve a serpentine layout. The parallel lines 852 may be treated as being divided into M groups each having N adjacent parallel lines. In the illustrated embodiment, there are eight groups (M=8) 852a-h each having eight (N=8) adjacent parallel lines, for a total of sixty-four (64) parallel lines 852. Each group of parallel lines 852a-h can be connected to one of the first plurality of metal pads 854 and to one of second plurality of metal pads 856, with the terminal metal pads (e.g., pads 854a and 854e) both connected to a single group (e.g., groups 852a and 852h, respectively) and the non-terminal metal pads (e.g., metal pads 854b-d, 856a-d) each being connected to two adjacent groups of parallel lines. In this arrangement, current can flow between the two terminals 854a, 854e following a serpentine layout, as illustrated by alternating arrows in the figure.

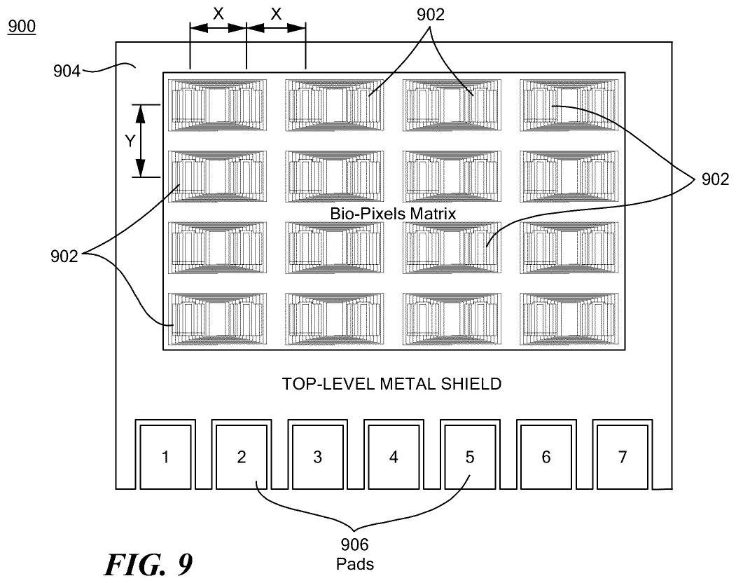

FIG. 9 shows an example sensor IC package 900 having a matrix of bio pixels 902 for analyzing bio samples. A shield 904 may be placed around the pixel matrix where no fluid will be applied. The shield 904 works as a heat sink to dissipate heat before transfer toward the pixels 902 where bio functionalization is located. Electronic circuitry on a die may be located under the shield 904. In addition, IC pads 906 are relatively close to the active electronic circuitry. The shield 904 brings heat toward the pads 906 and bonding.

It is understood that any practical number of pixels and accompanying coil configurations can be used to meet the needs of a particular application.

As used herein, the term “magnetic field sensing element” is used to describe a variety of electronic elements that can sense a magnetic field. The magnetic field sensing element can be, but is not limited to, a Hall effect element, a magnetoresistance (MR) element, or a magnetotransistor. As is known, there are different types of Hall effect elements, for example, a planar Hall element, a vertical Hall element, and a Circular Vertical Hall (CVH) element. As is also known, there are different types of MR elements, for example, a semiconductor magnetoresistance element such as Indium Antimonide (InSb), a giant magnetoresistance (GMR) element, for example, a spin valve, an anisotropic magnetoresistance element (AMR), a tunneling magnetoresistance (TMR) element, and a magnetic tunnel junction (MTJ). The magnetic field sensing element may be a single element or, alternatively, may include two or more magnetic field sensing elements arranged in various configurations, e.g., a half bridge or full (Wheatstone) bridge. Depending on the device type and other application requirements, the magnetic field sensing element may be a device made of a type IV semiconductor material such as Silicon (Si) or Germanium (Ge), or a type III-V semiconductor material like Gallium-Arsenide (GaAs) or an Indium compound, e.g., Indium-Antimonide (InSb).

As is known, some of the above-described magnetic field sensing elements tend to have an axis of maximum sensitivity parallel to a substrate that supports the magnetic field sensing element, and others of the above-described magnetic field sensing elements tend to have an axis of maximum sensitivity perpendicular to a substrate that supports the magnetic field sensing element. In particular, planar Hall elements tend to have axes of sensitivity perpendicular to a substrate, while metal based or metallic MR elements (e.g., GMR, TMR, AMR) and vertical Hall elements tend to have axes of sensitivity parallel to a substrate.

As used herein, the term “magnetic field sensor” is used to describe an assembly that uses a magnetic field sensing element in combination with an electronic circuit, all disposed upon a common substrate, e.g., a semiconductor substrate. Magnetic field sensors are used in a variety of applications, including, but not limited to, an angle sensor that senses an angle of a direction of a magnetic field, a current sensor that senses a magnetic field generated by a current carried by a current-carrying conductor, a magnetic switch that senses the proximity of a ferromagnetic object, a rotation detector that senses passing ferromagnetic articles, for example, magnetic domains of a ring magnet or a ferromagnetic target (e.g., gear teeth) where the magnetic field sensor is used in combination with a back-biased or other magnet, and a magnetic field sensor that senses a magnetic field density of a magnetic field.

Various embodiments of the concepts systems and techniques are described herein with reference to the related drawings. Alternative embodiments can be devised without departing from the scope of the described concepts. It is noted that various connections and positional relationships (e.g., over, below, adjacent, etc.) are set forth between elements in the claims, detailed description, and drawings. These connections and/or positional relationships, unless specified otherwise, can be direct or indirect, and the claimed inventions are not intended to be limiting in this respect. Accordingly, a coupling/connection of entities can refer to either a direct or an indirect coupling/connection, and a positional relationship between entities can be a direct or indirect positional relationship. As an example of an indirect positional relationship, references in the present description to element or structure A coupled/connected to element or structure B include situations in which one or more intermediate elements or structures (e.g., element C) is provided between elements A and B regardless of whether the characteristics and functionalities of elements A and/or B are substantially changed by the intermediate element(s).

Furthermore, it should be appreciated that relative, directional or reference terms (e.g. such as “above,” “below,” “left,” “right,” “top,” “bottom,” “vertical,” “horizontal,” “front,” “back,” “rearward,” “forward,” etc.) and derivatives thereof are used only to promote clarity in the description of the figures. Such terms are not intended as, and should not be construed as, limiting. Such terms may simply be used to facilitate discussion of the drawings and may be used, where applicable, to promote clarity of description when dealing with relative relationships, particularly with respect to the illustrated embodiments. Such terms are not, however, intended to imply absolute relationships, positions, and/or orientations. For example, with respect to an object or structure, an “upper” or “top” surface can become a “lower” or “bottom” surface simply by turning the object over. Nevertheless, it is still the same surface and the object remains the same.

The terms “disposed over,” “overlying,” “atop,” “on top,” “positioned on” or “positioned atop” mean that a first element, such as a first structure, is present on a second element, such as a second structure, where intervening elements or structures (such as an interface structure) may or may not be present between the first element and the second element. The term “direct contact” means that a first element, such as a first structure, and a second element, such as a second structure, are connected without any intermediary elements or structures between the interface of the two elements. The term “connection” can include an indirect connection and a direct connection.

The terms “parallel” and “perpendicular” are used in various contexts herein. It should be understood that the terms parallel and perpendicular do not require exact perpendicularity or exact parallelism, but instead it is intended that normal manufacturing tolerances apply, which tolerances depend upon the context in which the terms are used. In some instances, the term “substantially” is used to modify the terms “parallel” or “perpendicular.” In general, use of the term “substantially” reflects angles that are beyond manufacturing tolerances, for example, within +/−ten degrees.

In the foregoing detailed description, various features are grouped together in one or more individual embodiments for the purpose of streamlining the disclosure. This method of disclosure is not to be interpreted as reflecting an intention that each claim requires more features than are expressly recited therein. Rather, inventive aspects may lie in less than all features of each disclosed embodiment.

References in the disclosure to “one embodiment,” “an embodiment,” “some embodiments,” or variants of such phrases indicate that the embodiment(s) described can include a particular feature, structure, or characteristic, but every embodiment can include the particular feature, structure, or characteristic. Moreover, such phrases are not necessarily referring to the same embodiment(s). Further, when a particular feature, structure, or characteristic is described in connection knowledge of one skilled in the art to affect such feature, structure, or characteristic in connection with other embodiments whether or not explicitly described.

The disclosed subject matter is not limited in its application to the details of construction and to the arrangements of the components set forth in the detailed description or illustrated in the drawings. The disclosed subject matter is capable of other embodiments and of being practiced and carried out in various ways. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

Other variations to the disclosed embodiments can be understood and effected by those skilled in the art in practicing the claimed invention, from a study of the drawings, the disclosure, and the appended claims.

The mere fact that certain measures are recited in mutually different dependent claims does not indicate that a combination of these measures cannot be used to obtain an advantage. Any reference signs in the claims should not be construed as limiting the scope.

All publications and references cited herein are expressly incorporated herein by reference in their entirety.

What is claimed is:

1. A magnetic field bio sensor, comprising:
a first coil having first and second portions on different metal layers, wherein the first and second portions of the first coil are connected by first vias; and
a second coil having first and second portions on different ones of the metal layers, wherein the first and second portions of the second coil are connected by second vias,
wherein the second portion of the first coil overlaps with the first portion of the second coil to promote heat dissipation via an inactive one of the first and second coils.

2. The sensor according to claim 1, wherein the first portions of the first and second coils are formed on a first one of the metal layers.

3. The sensor according to claim 1, wherein the first coil is on multiples ones of the metal layers.

4. The sensor according to claim 2, wherein the second portions of the first and second coils are formed on a second one of the metal layers.

5. The sensor according to claim 4, wherein the second coil is on multiples ones of the metal layers.

6. The sensor according to claim 1, wherein the first coil includes an active area in which a first portion of the first coil splits into N segments.

7. The sensor according to claim 6, wherein the N segments are parallel to each other.

8. The sensor according to claim 6, wherein the N segments recombine.

9. The sensor according to claim 6, wherein N is between 2 and 100 inclusive.

10. The sensor according to claim 6, wherein the active area is configured to sense return from at least one MR element proximate a bio sample.

11. The sensor according to claim 1, wherein the overlap of the second portion of the first coil overlaps and the first portion of the second coil is configured to dissipate heat generated by the first coil via heat transfer in the second coil.

12. The sensor according to claim 1, wherein the sensor comprises an IC package having bio pixels for respective samples of the bio material.

13. The sensor according to claim 12, wherein the first coil provides first and second pixels.

14. A method, comprising:
forming a first coil having first and second portions on different metal layers, wherein the first and second portions of the first coil are connected by first vias; and
forming a second coil having first and second portions on different ones of the metal layers, wherein the first and second portions of the second coil are connected by second vias, wherein the first and second coils form part of a magnetic field bio sensor,
wherein the second portion of the first coil overlaps with the first portion of the second coil to promote heat dissipation via an inactive one of the first and second coils.

15. The method according to claim 14, wherein the first portions of the first and second coils are formed on a first one of the metal layers.

16. The method according to claim 14, wherein the first coil is on multiples ones of the metal layers.

17. The method according to claim 15, wherein the second portions of the first and second coils are formed on a second one of the metal layers.

18. The method according to claim 17, wherein the second coil is on multiples ones of the metal layers.

19. The method according to claim 14, wherein the first coil includes an active area in which a first portion of the first coil splits into N segments.

20. The method according to claim 19, wherein the N segments are parallel to each other.

21. The method according to claim 19, wherein the N segments recombine.

22. The method according to claim 19, wherein N is between 2 and 100 inclusive.

23. The method according to claim 19, wherein the active area is configured to sense return from at least one MR element proximate a bio sample.

24. The method according to claim 14, wherein the overlap of the second portion of the first coil overlaps and the first portion of the second coil is configured to dissipate heat generated by the first coil via heat transfer in the second coil.

25. The method according to claim 14, wherein the first coil provides first and second pixels.
